(19) United States
(12) Patent Application Publication
(10) Pub. No.: US 2018/0002657 A1
(43) Pub. Date: Jan. 4, 2018

(54) PROCESS FOR ENRICHING THE BIOMASS OF MICROALGAE OF THE THRAUSTOCHYTRIUM GENUS WITH DHA AND WITH ARG AND GLU AMINO ACIDS

(71) Applicant: Roquette Freres, Lestrem (FR)

(72) Inventor: Bernard Caulier, Fretin (FR)

(73) Assignee: Roquette Freres, Lestrem (FR)

(21) Appl. No.: 15/546,606

(22) PCT Filed: Jan. 26, 2016
(86) PCT No.: PCT/FR2016/050159

§ 371 Date: Jul. 26, 2017

(30) Foreign Application Priority Data
Jan. 27, 2015 (FR) 15 50598

(51) Int. Cl. C12N 1/12

(52) U.S. Cl. CPC C12N 1/12

(57) ABSTRACT

The present invention relates to a process for enriching a biomass of microalgae of the Thraustochytrium genus with DHA and with arginine and glutamic acid amino acids, characterized in that it comprises a step aimed at limiting the rate of growth of the microalga while at the same time maintaining or continuously introducing a source of nitrogen in or into the fermentation medium.

The present invention relates to a novel fermentative process for enriching the biomass of microalgae of the Thraustochytrium genus, more particularly Schizochytrium sp. or Schizochytrium mangrovei, with docosahexanoic acid (DHA) and with arginine and glutamic acid amino acids, and also to a process for producing the oil extracted from this microalgal biomass.

Technical Field of Lipids

Lipids constitute one of the three major families of macronutrients with proteins and carbohydrates.

Among the lipids, triglycerides and phospholipids in particular stand out:
Triglycerides (also called triacylglycerols or triacylglycerides or TAGs) are glycerides in which the three hydroxyl groups of the glycerol are esterified with fatty acids. They are the main constituent of vegetable oil and of animal fats.

Triglycerides represent approximately 95% of the dietary lipids ingested by humans. In the organism, they are present mainly in adipose tissues and constitute the main form of energy storage.
Phospholipids are amphiphilic lipids, that is to say lipids consisting of a polar (hydrophilic) “head” and two aliphatic (hydrophobic) “tails”.

Phospholipids are structural lipids since they are constituents of cell membranes for which they provide, inter alia, the fluidity.

Triglycerides and phospholipids are composed predominantly of fatty acids which are both provided by the diet and, for some of them, synthesized by the organism.

The biochemical classification (based on the number of double bonds contained in the fatty acid molecule) distinguishes saturated fatty acids (SFAs), monounsaturated fatty acids (MUFAs) and polyunsaturated fatty acids (PUFAs).

From the physiological point of view, the following are distinguished:
indispensable fatty acids, required for the development and correct functioning of the human body, but which our body is not able to produce;
“conditionally” indispensable fatty acids which are essential for normal growth and the physiological functions of cells, but which can be produced from their precursor if it is provided by the diet. They are therefore absolutely required if their essential precursor is absent;
non-indispensable fatty acids.

All the indispensable and “conditionally” indispensable fatty acids constitute essential fatty acids.

The other fatty acids are referred to as non-essential.

The non-indispensable fatty acids include, in particular:
eicosapentaenoic acid (EPA) of the omega 3 fatty acid family,
oleic acid, the predominant monounsaturated fatty acid in our diet, and palmitoleic acid,
saturated fatty acids, such as lauric acid, myristic acid or palmitic acid.

More particularly, polyunsaturated fatty acids are classified according to the position of the first double bond, starting from the final methyl function.

Thus, in the nomenclature, for omega “x” or “nx”, “x” corresponds to the position of the first unsaturation.

Two major families of essential fatty acids are distinguished: omega 6 fatty acids (or n-6 PUFAs), of which the precursor and the major representative is linoleic acid (LA), and omega 3 fatty acids (or n-3 PUFAs), of which the precursor is alpha-linolenic acid (ALA).

The majority of the polyunsaturated fatty acids of biological interest belong to the omega 6 family (arachidonic acid or ARA) or omega 3 family (eicosapentaenoic acid or EPA, docosahexaenoic acid or DHA).

In addition, in the nomenclature, the number of carbons constituting the chain is also defined: thus, EPA is described as C20:5 and DHA as C22:6.

The “5” and “6” thus correspond to the number of unsaturations of the carbon chain presented respectively by EPA and by DHA.

DHA, of the omega 3 fatty acid family, is a fatty acid that the organism knows how to synthesize from alpha-linolenic acid, or which is provided by the consumption of oily fish (tuna, salmon, herring, etc.).

DHA plays an important role in the structure of membranes and in the development and function of the brain and of the retina.

Fish oils are used mainly as a source of omega 3 fatty acids, such as DHA and EPA, but they are also found in oils of microalgae, from which they are extracted either as a mixture, or separately, as is the case for example with the oils derived from certain selected strains, such as those of the genus Schizochytrium, which contain only traces of EPA but high DHA contents.

Technical Field of Peptides and Amino Acids

Peptides and amino acids are conventionally exploited as functional agents or food supplements in many fields.

In the context of supplying amino acids of interest, it may in fact be advantageous to have available peptide sources that are rich in arginine and glutamic acid.

Arginine is an amino acid that has many functions in the animal kingdom.

Arginine may be degraded and may thus serve as a source of energy, carbon and nitrogen for the cell which assimilates it.

In various animals, including mammals, arginine is decomposed into ornithine and urea. The latter is a nitrogenous molecule that can be eliminated (via excretion in the urine) so as to regulate the amount of nitrogenous compounds present in the cells of animal organisms.

Arginine allows the synthesis of nitrogen monoxide (NO) via NO synthetase, thus participating in the vasodilation of the arteries, which reduces the rigidity of the blood vessels, increases the blood flow and thus improves the functioning of the blood vessels.

Food supplements which contain arginine are recommended for promoting the health of the heart, the vascular function, for preventing “platelet aggregation” (risk of formation of blood clots) and for lowering the arterial pressure.

The involvement of arginine in the healing of wounds is associated with its role in the formation of proline, which is another important amino acid in collagen synthesis.

Finally, arginine is a component that is frequently used, in particular by sportspeople, in energy drinks.

As regards glutamic acid, it is not only one of the elementary bricks used for protein synthesis, but is also the excitatory neurotransmitter that is the most widespread in the central nervous system (encephalon+spinal column) and is a GABA precursor in GABAergic neurons.

Under the code E620, glutamate is used as a flavor enhancer in foods. It is added to food preparations to enhance their taste.

Besides glutamate, the Codex Alimentarius has also recognized as flavor enhancers the sodium salt (E621), the potassium salt (E622), the calcium salt (E623), the ammonium salt (E624) and the magnesium salt (E625) thereof.

Glutamate (or the salts thereof) is often present in ready-made meals (soups, sauces, crisps and ready-made dishes). It is also commonly used in Asian cookery.

It is currently frequently used in combination with flavorings in aperitifs (bacon flavor, cheese flavor). This makes it possible to enhance the bacon, cheese, etc. flavor. It is rare to find an aperitif not containing any.

It is also found in certain medicament capsules, but not for its taste functions.

Finally, it is the major component of cooking auxiliaries (stock cubes, sauce bases, sauces, etc.).

Production of Lipids, in Particular of Fatty Acids, by Microalgae

Microalgae of the genus Schizochytrium are conventionally cultured in fermenters (heterotrophic conditions: in darkness and in the presence of a carbon source).

It should be noted that the profitable utilization of these microalgae generally requires controlling the fermentation conditions.

To achieve this result, first processes for fermentation making it possible to obtain high cell densities (HCDs) have thus been greatly developed in order to obtain maximum lipid yields and productivities.

The aim of these HCD cultures was to obtain the highest possible concentration of the desired lipids in the shortest period of time possible.

However, it quickly became apparent to specialists in the field that it is necessary for example to subject the microalgae to a nutritional stress which limits their growth, when it is desired to make them produce large lipid stores.

Therefore, growth and production are conventionally uncoupled in fermenting processes.

For example, to promote the accumulation of polyunsaturated fatty acids (in this instance docosahexaenoic acid or DHA), patent application WO 01/54510 recommends dissociating cell growth from the production of polyunsaturated fatty acids.

More particularly, a process for producing microbial lipids is claimed, which process comprises the steps consisting in:

(a) carrying out fermentation of a medium comprising microorganisms, a carbon source and a limiting nutritional source, and ensuring conditions sufficient to maintain a dissolved oxygen content of at least approximately 4% of saturation in said fermentation medium to increase the biomass;

(b) then providing conditions sufficient to maintain a dissolved oxygen content of approximately less than or equal to 1% of saturation in said fermentation medium and providing conditions sufficient to allow said microorganisms to produce said lipids;

(c) and collecting said microbial lipids, in which at least approximately 15% of said microbial lipids are constituted of polyunsaturated lipids;

and in which a biomass density of at least approximately 100 g/l is obtained over the course of the fermentation.

In the microalga Schizochytrium sp. strain ATCC 20888, a first growth phase is thus more particularly performed in the presence of a carbon source and a nitrogen source but without limiting oxygen, so as to promote the production of a high cell density, then, in a second phase, the supply of nitrogen is stopped and the supply of oxygen is gradually slowed (management of the dissolved oxygen pressure or pO₂ from 10% to 4% then to 0.5%), so as to stress the microalga, slow its growth and trigger production of the fatty acids of interest.

In the microalga Ctypthecodinium cohnii, the higher DHA content is obtained at low glucose concentration (of the order of 5 g/l) and thus at a low growth rate (Jiang and Chen, 2000, Process Biochem., 35(10), 1205-1209).

Consequently, in the event that the formation of products is not correlated with high cell growth, it is taught that it is prudent to control the rate of cell growth.

In general, those skilled in the art choose to control the growth of the microalgae by controlling the fermentation conditions (temperature, pH, etc.) or by regulated feeding of nutritional components to the fermentation medium (semicontinuous conditions referred to as “fed batch”).

If they choose to control the growth of the microalgae heterotrophically through the supply of carbon sources, those skilled in the art generally choose to adapt the carbon source (pure glucose, acetate, ethanol, etc.) to the microalga (C. cohnii, Euglena gracilis, etc.) as a function of the metabolite produced (for example a polyunsaturated fatty acid of DHA type).

Temperature may also be a key parameter. For example, it has been reported that the synthesis of polyunsaturated fatty acids in some species of microalgae, such as EPA by Chlorella minutissima, is promoted at a lower temperature than that required for the optimal growth of said microalga.

To optimize the production of triglycerides, those skilled in the art are also led to optimize the carbon flow toward oil production, by acting on the nutritional environment of the fermentation medium.

Thus, it is known that oil accumulates when there is a sufficient supply of carbon but under conditions of nitrogen deficiency.

Therefore, the C/N ratio is the determining factor here, and it is accepted that the best results are obtained by acting directly on the nitrogen content, with the glucose content not being a limiting factor.

To optimize oil production, it is therefore essential for those skilled in the art to control the carbon flow by moving it toward oil production to the detriment of protein production; the carbon flow is redistributed and accumulates as lipid storage substances when the microalgae are placed in a nitrogen-deficient medium.

Production of Proteins by Microalgae

As explained in detail above, to optimize the production of triglycerides, those skilled in the art are led to optimize the carbon flow toward oil production, by acting on the nutritional environment of the fermentation medium.

In a study carried out in a microalga of Chlorella type, it has been noted that a nitrogen deficiency affects cell growth, thereby resulting in a growth rate reduced by 30% compared with the normal growth rate of the microalga (Xiong et al., Plant Physiology, 2010, 154, pp. 1001-1011).

To explain this result, in the abovementioned article Xiong et al. in fact demonstrate that if the Chlorella biomass is divided into its 5 main components, in particular carbohydrates, lipids, proteins, DNA and RNA (representing 85% of the solids thereof), then the C/N ratio has no impact on the content of DNA, RNA or carbohydrates, but it becomes paramount for the content of proteins and lipids.

Thus, Chlorella cells cultivated with a low C/N ratio contain 25.8% proteins and 25.23% lipids, whereas a high C/N ratio makes the synthesis of 53.8% lipids and 10.5% proteins possible.

To optimize protein production, it is therefore essential for those skilled in the art to control the carbon flow by moving it toward protein production to the detriment of lipid production; the carbon flow is redistributed and accumulates as protein storage substances when the microalgae are placed in a medium that is not nitrogen deficient.

Armed with this teaching, in order to produce biomasses that are rich in proteins and thus in amino acids which constitute them, those skilled in the art are therefore led to work the fermentation conditions by instead promoting a low C/N ratio, and thus:
supply a large amount of nitrogen source to the fermentation medium while keeping constant the carbon source feedstock, which will be converted into proteins, and
stimulate the growth of the microalga.

### SUMMARY OF THE INVENTION

The present invention relates to a process for producing a biomass of microalgae of the Thraustochytrium genus of which the lipid fraction is rich in DHA and of which the content of arginine and glutamic acid amino acids relative to total amino acids is high.

This process is based on the control of the growth rate of the microalga, this control being exerted so as to reduce it to its minimum, while at the same time maintaining or continuously introducing a nitrogen source in or into the fermentation medium.

This result can for example be obtained by reducing or exhausting trace elements in the fermentation medium or by limiting O₂ transfer.

In one preferential embodiment of the process in accordance with the invention, it is thus chosen to limit the growth rate of the microalga by limiting the oxygen supply.

For the purposes of the invention, the limitation of the growth rate is assessed by the ratio between the actual growth rate of the microalga (μ) with regard to its optimal growth rate (μmax), where “ρ” is the speed of growth expressed in g of biomass formed per g of biomass and per hour, that is to say (h⁻¹).

More particularly, the process of the invention is a process for enriching a biomass of microalgae of the Thraustochytrium genus with DHA and with arginine and glutamic acid amino acids, characterized in that it comprises a step consisting in maintaining or adding a nitrogen source in or to the fermentation medium as soon as the value of the ratio of the growth rates μ/μmax of the microalgae becomes less than 0.2.

Preferably, the microalgae are of the genus Schizochytrium sp. or Schizochytrium mangrovei.

More specifically, the microalgae may be a strain selected from the strains CNCM I-4469 and CNCM I-4702 deposited with the Collection Nationale de Cultures de Microorganismes [French National Collection of Microorganism Cultures] of the Institut Pasteur on Apr. 14, 2011 and Nov. 22, 2012, respectively.

Optionally, the process may also comprise harvesting the biomass, optionally preparing a cell extract or lysate from this biomass, then optionally extracting a crude oil rich in DHA and in arginine and glutamic acid amino acids.

The process according to the present invention may be characterized in that the biomass obtained comprises:
at least 45% of DHA by weight of total fatty acids; and
at least 10% of arginine and at least 25% of glutamic acid by weight relative to total amino acids, preferably at least 15% of arginine and at least 40% of glutamic acid by weight relative to total amino acids.

### DETAILED DESCRIPTION OF THE INVENTION

Within the context of the invention, the applicant company has chosen to explore an original route for optimizing the production of DHA and of arginie and glutamic acid amino acids by proposing a novel way of conducting fermentation.

The applicant company has thus found, which goes against the technical preconceptions on the subject, that it is possible to produce by fermentation microalgal biomasses:
rich in lipids (more than 25% by dry weight of biomass, preferably at least 30%), the predominant fatty acid of which is docosahexaenoic acid (DHA), and
rich in arginine and glutamic acid amino acids (more than 35% by weight of the total amino acids, preferably at least 55%),

without it being essential, as described in the prior art, to maximize the C/N ratio (consumed carbon to consumed nitrogen, mole/mole).

The applicant company has thus found that it is possible to modify the lipid and amino acid composition of the biomass produced by fermentation, through the maintaining, which is not conventional for a lipid production, of the nitrogen feed throughout the fermentation even when the growth rate μ/μmax is less than 0.2.

Indeed, the applicant company has understood that, when the μ/μmax ratio becomes less than 0.2, following a limitation of a nutritive substrate other than the nitrogenous or carbon-based substrates, it is possible to move the metabolic productions toward the production of arginine and glutamic acid amino acids, while at the same time retaining a considerable DHA production.

In one embodiment, the limitation which makes it possible to reduce the growth rate can be the limitation of the oxygen supply (OTR, oxygen transfer rate).

In particular, the OTR during the fermentation phase is preferably from 30 to 35 mmol/l/h.

The growth limitation can also be induced by exhausting trace elements or minerals, preferably chosen from phosphate, magnesium or potassium.

More particularly, the applicant company has found that it is necessary to supply nitrogen, preferentially in aqueous ammonia form (used for example in pH regulation), or that it is necessary to maintain the nitrogen supply, until the end of the culture, provided that μ is less than 20% of μmax.

In one preferred embodiment, the initial nitrogen supply is added to by the regulation of the pH, the nitrogen consumed thus being compensated for by that of the regulation of the pH. This makes it possible to obtain a C/N ratio (consumed carbon to consumed nitrogen, mole/mole) at the end of the culture of less than 20, for example of between 10 and 15, and preferably of approximately 15.

The strains to be used in the methods of the present invention are of the Thraustochytrium genus, more particularly Schizochytrium mangrovei or Schizochytrium sp. Such strains are known to those skilled in the art.

In the course of their research, the applicant company has identified several microalgal strains of great interest which produce DHA. The applicant company is especially quite particularly interested in two strains that it has identified.

The first strain is a strain of Schizochytrium sp., deposited in France on Apr. 14, 2011 with the Collection Nationale de Cultures de Microorganismes [French National Collection of Microorganism Cultures] (CNCM) of the Institut Pasteur, 25 rue du Docteur Roux, 75724 Paris Cedex 15, France, under number I-4469 and also in China with the China Center for Type Culture Collection (CCTCC) of the University of Wuhan, Wuhan 430072, P.R. China under number M 209118. This strain mainly produces DHA and to a lesser extent palmitic acid and palmitoleic acid. It was characterized by partial sequencing of the gene encoding 188 RNA (SEQ ID No 1):

| | |
|---|---|
| 1 | GAGGGTTTTA CATTGCTCTC aTTCCaATAG CAaGACGCGA AGCGCCCCGC ATTGATATTT |
| 61 | CTCGTCACTA CCTCGTGGAG TCCACATTGG GTAATTTACG CGCCTGCTGC CTTCCTTGGA |
| 121 | TGTGGTAGCC GTCTCTCAGG CTCCCTCTCC GGAGTCGAGC CCTAACTCCC CGTCACCCGT |
| 181 | TATAGTCACC GTAGGCCAAT ACCCTACCGT CGACAACTGA TGGGGCAGAA ACTCAAACGA |
| 241 | TTCATCGCTC CGAAAAGCGA TCTGCTCAAT TATCATGACT CACCAAGAGA GTTGGCTTAG |
| 301 | ACCTAATAAG TGCGGCCCTC CCCGAAAGTC GGGCCCGTAC AGCACGTATT AATTCCAGAA |
| 361 | TTACTGCAGG TATCCGTATA AAGGAACTAC CGAAGGGATT ATAACTGATA TAATGAGCCG |
| 421 | TTCGCAGTTT CACAGTATAA TTCGCTTATA CTTACACATG CATGGCTTAG TCTTTGAGA |

which made it possible to identify it as being a strain of Schizochytrium sp. type. This strain will be subsequently denoted “CNCM I-4469” in the present application.

Moreover, the second strain is a strain of Schizochytrium mangrovei. It produces DHA and palmitic acid in relatively equal proportions. It was deposited by the applicant company in France on Nov. 22, 2012 with the Collection Nationale de Cultures de Microorganismes (CNCM) of the Institut Pasteur, 25 rue du Docteur Roux, 75724 Paris Cedex 15, under number CNCM I-4702. It was characterized by sequencing of the genes encoding 18 S rRNA (SEQ ID No 2):

| | |
|---|---|
| 1 | GGTTTTACAT TGCTCTCATT CCGATAGCAA AACGCATACA CGCTTCGCAT CGATATTTCT |
| 61 | CGTCCTACCT CGTGGAGTCC ACAGTGGGTA ATTTACGCGC CTGCTGCTAT CCTTGGATAT |
| 121 | GGTAGCCGTC TCTCAGGCTC CCTCTCCGGA GTCGAGCCCT AACTCTCCGT CACCCGTTAT |
| 181 | AGTCACCGTA GTCCAATACA CTACCGTCGA CAACTGATGG GGCAGAAACT CAAACGATTC |
| 241 | ATCGACCAAA AWAGTCAATC TGCTCAATTA TCATGATTCA CCAATAAAAT CGGCTTCAAT |
| 301 | CTAATAAGTG CAGCCCCATA CAGGGCTCTT ACAGCATGTA TTATTTCCAG AATTACTGCA |
| 361 | GGTATCCATA TAAAAGAAAC TACCGAAGAA ATTATTACTG ATATAATGAG CCGTTCGCAG |
| 421 | TCTCACAGTA CAATCGCTTA TACTTACACA GCAG |

which made it possible to identify it as being a strain of Schizochytrium mangrovei type. This strain will be subsequently denoted “CNCM I-4702” in the present application.

Moreover, the fermenting processes according to the present invention are carried out under heterotrophic culturing conditions. These conditions adapted to the microalgae under consideration and also the culture media are well known to those skilled in the art.

The carbon source necessary for the growth of the microalga is preferably glucose.

Preferably, the glucose supply is such that the glucose concentration during the fermentation is maintained at a concentration of 20 g/l or more. At the end of fermentation, the glucose concentration is at least 5 g/l.

The nitrogen source may be extracts of yeast, urea, sodium glutamate, ammonium sulfate, aqueous ammonia with pH regulation, used alone or in combination.

Generally, the culturing step comprises a preculturing step to revive the strain, then a step of culturing or fermentation proper. The latter step corresponds to the step of production of the lipids of interest, in particular of DHA.

Preferably, the pH is regulated during the fermentation at a pH of between 5 and 7, preferably approximately 6.

Preferably, the temperature during the fermentation is 26-30° C., preferably approximately 28° C.

The fermentation time is preferably at least 50 hours, preferably between 65 and 90 hours, even more preferably between 70 and 85 hours.

The fermentation process according to the present invention makes it possible to obtain (or is carried out in such a way as to obtain) a biomass comprising at least 45% of DHA by weight of total fatty acids. In addition, the process guarantees a lipid content by weight relative to the biomass of at least 25%. Thus, the biomass is indeed enriched with DHA.

Moreover, the fermentation process according to the present invention makes it possible to obtain (or is carried out in such a way as to obtain) a biomass comprising at least 40% of proteins by weight relative to the biomass. In addition, the proportion of glutamic acid relative to the total amino acids is at least 25%. The arginine proportion is at least 10%.

For the CNCM I-4702 strain, the results obtained with the fermentation process according to the invention are a biomass comprising approximately 47% of DHA by weight of total fatty acids, with a lipid content by weight relative to the biomass of approximately 35%, and approximately 53% of proteins with a proportion of glutamic acid of approximately 40% and of arginine of approximately 16%.

For the CNCM I-4469 strain, the results obtained with the fermentation process according to the invention are a biomass comprising approximately 52% of DHA by weight of total fatty acids, with a lipid content by weight relative to the biomass of approximately 26%, and approximately 43% of proteins with a proportion of glutamic acid of approximately 26% and of arginine of approximately 10%.

When reference is made to a percentage by weight, it is understood to be by dry weight.

Aside from the biomass, the present invention also relates to a cell extract or lysate prepared from this biomass. In particular, this extract or lysate is prepared from the biomass recovered after fermentation. This extract or lysate is rich in DHA and in arginine and glutamic acid amino acids.

The cells may be ruptured to extract the lipid content in various ways, including mechanical, chemical and enzymatic ways.

An oil can subsequently be extracted from the cell lysate.

Thus, the method for producing lipids of interest, preferably DHA, and arginine and glutamic acid amino acids, comprises the fermenting process according to the present invention, harvesting the biomass, preparing a cell extract or lysate and extracting a crude oil comprising the lipids of interest, preferably DHA and optionally arginine and glutamic acid amino acids.

The term “approximately” is intended to mean the value + or −10% of said value, preferably + or −5% of said value.

The invention will be understood more clearly from the following examples which are intended to be illustrative and nonlimiting.

### EXAMPLES

### Example 1: Conditions for Culturing the CNCN I-4702 Strain

The protocol comprises preculturing for inoculation of the fermenter at 0.1 g/l of biomass for the Schizochytrium mangrovei CNCM I-4702 strain.

Preculturing

The preculturing (100 ml of medium) in a 500 ml baffled Erlenmeyer flask lasts for 24 h at 28° C.

All of the components of the medium are sterilized by filtration.

TABLE I

| Preculture medium | % (g/g) |
|---|---|
| Anhydrous glucose | 3 |
| Yeast extract | 0.4 |
| Monosodium glutamate | 6.42 |
| NaCl | 1.25 |
| MgSO₄•7(H₂O) | 0.4 |
| KCl | 0.05 |
| CaCl₂•2(H₂O) | 0.01 |
| NaHCO₃ | 0.05 |
| KH₂PO₄ | 0.4 |
| Stock solution vitamins B1, B6, B12 | 0.1 |
| Stock solution trace elements | 0.8 |

Culturing

The medium is sterilized in 3 parts.

The glucose is sterilized with the KH₂PO₄ for an addition just before T₀.

The remainder of the salts are sterilized in the fermenter with 0.75 ml/l of Clearol FBA 3107. The trace elements and vitamins are sterilized by filtration.

The volume at T₀ represents 75% of the final volume. The pH is adjusted at T₀ using aqueous ammonia, then it is regulated at 6, still with aqueous ammonia.

TABLE II

| Culture medium | % (W/W) |
|---|---|
| KH₂PO₄ | 0.80 |
| (NH₄)₂SO₄ | 0.33 |
| Na₂SO₄ | 0.67 |
| NaCl | 0.27 |
| CaCl₂•2(H₂O) | 0.03 |
| MgSO₄•7(H₂O) | 1.00 |
| Anhydrous glucose | 6.00 |
| Stock solution vitamins B1, B6, B12 | 0.20 |
| Stock solution trace elements | 0.27 |

A fed batch of glucose (concentration: 500 g/l) is supplied continuously starting from T₀ at a constant rate (to be adjusted according to calculations) so as not to be at a concentration lower than 20 g/l. At the end, the glucose will be exhausted without descending below 5 g/l at the time fermentation is stopped.

The culturing is carried out at 28° C. and lasts from 70 to 85 hours with a fixed and constant OTR (oxygen uptake rate) of 20 to 30 mmol of O₂/l/h.

Stock Solutions

| vitamins | g/l |
|---|---|
| B1 | 45 |
| B6 | 45 |
| B12 | 0.25 |

| Trace elements | g/l |
|---|---|
| MnCl₂•2H₂O | 8.60 |
| CoCl₂•6H₂O | 0.2 |
| NiSO₄•6H₂O | 7.50 |
| Na₂MoO₄•2H₂O | 0.15 |
| ZnSO₄•7H₂O | 5.70 |
| CuSO₄•5H₂O | 6.50 |
| FeSO₄•7H₂O | 32.00 |
| Zinc acetate | 0.01 |
| EDTA | Brought to pH >3 |

Two fermentation conditions are implemented:
As a control: “standard” conditions, in which the C/N ratio (consumed carbon to consumed nitrogen) is maximized so as to produce essentially lipids by interrupting the nitrogen supply but not that of the carbon-based substrate, this being without limitation of O₂. These conditions are thus nitrogen deficient. Suppression of the nitrogen supply takes place when one or more salts are exhausted. Actual growth is then impossible or very limited: the cell multiplication rate drops to the benefit of the lipid enrichment of the cells present. The overall mass of the cells increases but the number of cells changes little since the growth rate falls.
According to the invention: Conditions which make it possible to produce lipids rich in DHA with amino acids rich in arginine and glutamic acid by limiting the growth rate by limiting O₂ transfer such that μ drops to μ/μmax <0.2 rapidly, while at the same time maintaining the supply of glucose and nitrogen preferentially through regulation of the pH with aqueous ammonia.

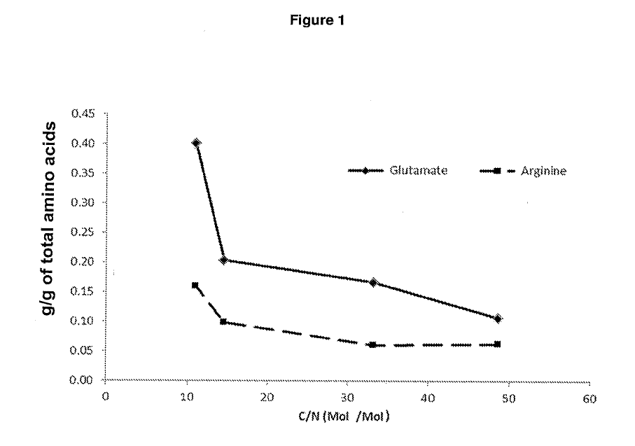

FIG. 1 presents the change in the proportion of arginine and glutamic acid among the amino acids as a function of the C/N calculated at the end of culture.

It appears that the process promotes the production of arginine and glutamic acid amino acids provided that the C/N ratio is less than 15 (# μ/μmax<0.2).

Table III below reflects, for the CNCN I-4702 strain, the fatty acid and amino acid composition of the biomass produced according to the “conventional” operating conditions and the operating conditions in accordance with the invention.

TABLE III

| | | Use of the |
|---|---|---|
| | Conventional | process of |
| | culture | the invention |
| Lipids relative to Biomass (g/g) | 0.60 | 0.35 |
| Proteins relative to Biomass according to N | 0.12 | 0.53 |
| 6.25 (g/g) | | |
| DHA/Fatty acids (g/g) | 0.24 | 0.47 |
| Aspartic Acid relative to Σ TAA (g/g) | 0.12 | 0.05 |
| Threonine relative to Σ TAA (g/g) | 0.06 | 0.03 |
| Serine relative to Σ TAA (g/g) | 0.06 | 0.03 |
| Glutamic Acid relative to Σ tAA (g/g) | 0.11 | 0.40 |
| Glycine relative to Σ TAA (g/g) | 0.05 | 0.03 |
| Alanine relative to Σ TAA (g/g) | 0.07 | 0.04 |
| Valine relative to Σ TAA (g/g) | 0.06 | 0.03 |
| Isoleucine relative to Σ TAA (g/g) | 0.05 | 0.03 |
| Leucine relative to Σ TAA (g/g) | 0.08 | 0.04 |
| Tyrosine relative to Σ TAA (g/g) | 0.04 | 0.02 |
| Phenylalanine relative to Σ TAA | 0.04 | 0.03 |
| (g/g) | | |
| Lysine relative to Σ TAA (g/g) | 0.07 | 0.04 |
| Histidine relative to Σ TAA (g/g) | 0.02 | 0.01 |
| Arginine relative to Σ TAA (g/g) | 0.06 | 0.16 |
| Proline relative to Σ TAA (g/g) | 0.05 | 0.03 |
| Cystine relative to Σ TAA (g/g) | 0.02 | 0.01 |
| Methionine relative to Σ TAA (g/g) | 0.03 | 0.02 |
| Tryptophan relative to Σ TAA (g/g) | 0.02 | 0.01 |

The glutamic acid proportion relative to the sum of the amino acids is multiplied by 3.75 and the arginine proportion relative to the sum of the amino acids is multiplied by 2.75.

The lipid composition is reduced, but the DHA content of the fatty acids is almost multiplied by two.

### Example 2: Conditions for Culturing the CNCN I-4469 Strain

The conditions for culturing this microalga are the same as those of example 1 (with the exception of the level of inoculum chosen in preculture, of about 5 g/l for Schizochytrium sp.).

According to, two culture conditions implemented “conventionally” and according to the invention.

Table IV below reflects the fatty acid and amino acid composition of the biomass produced according to the “conventional” operating conditions and the operating conditions in accordance with the invention.

TABLE IV

| | | Use of the |
|---|---|---|
| | | process |
| | Conventional | of the |
| | culture | invention |
| Lipids relative to Biomass (g/g) | 0.46 | 0.26 |
| Proteins relative to Biomass according to N | 0.21 | 0.43 |
| 6.25 (g/g) | | |
| DHA/Fatty acids (g/g) | 0.42 | 0.52 |
| Aspartic Acid relative to Σ TAA (g/g) | 0.12 | 0.08 |
| Threonine relative to Σ TAA (g/g) | 0.05 | 0.04 |
| Serine relative to Σ TAA (g/g) | 0.05 | 0.04 |
| Glutamic Acid relative to Σ tAA (g/g) | 0.15 | 0.26 |
| Glycine relative to Σ TAA (g/g) | 0.05 | 0.05 |
| Alanine relative to Σ TAA (g/g) | 0.08 | 0.06 |
| Valine relative to Σ TAA (g/g) | 0.06 | 0.05 |
| Isoleucine relative to Σ TAA (g/g) | 0.04 | 0.03 |
| Leucine relative to Σ TAA (g/g) | 0.08 | 0.06 |
| Tyrosine relative to Σ TAA (g/g) | 0.04 | 0.03 |
| Phenylalanine relative to Σ TAA (g/g) | 0.04 | 0.03 |
| Lysine relative to Σ TAA (g/g) | 0.06 | 0.05 |
| Histidine relative to Σ TAA (g/g) | 0.02 | 0.02 |
| Arginine relative to Σ TAA (g/g) | 0.06 | 0.10 |
| Proline relative to Σ TAA (g/g) | 0.04 | 0.07 |
| Cystine relative to Σ TAA (g/g) | 0.02 | 0.01 |
| Methionine relative to Σ TAA (g/g) | 0.02 | 0.02 |
| Tryptophan relative to Σ TAA (g/g) | 0.02 | 0.01 |

For the Schizochytrium sp. strain, the effects are identical but smaller. Moreover, an increase of 75% in the proportion of proline among the amino acids is also noted.

The arginine and glutamic acid amino acid contents increase respectively by 60% and 75%, while the protein content doubles.

The DHA content in the fatty acids increases by 23%.

1- A process for enriching a biomass of microalgae of the Thraustochytrium genus with docosahexaenoic acid (DHA) and with arginine and glutamic acid amino acids, comprising limiting the growth rate of the microalga in a fermentation medium, determining a ratio of growth rates μ/μmax of the microalgae, concurrently with said limiting maintaining or continuously introducing a nitrogen source in or into the fermentation medium as soon as the value of the ratio of the growth rates μ/μmax of the microalgae becomes less than 0.2, to produce a biomass.

2- The process according to claim 1, wherein the microalgae are of the genus Schizochytrium sp. or Schizochytrium mangrovei genus.

3- The process according to claim 1, wherein the microalgae are a strain selected from strain references CNCM I-4469 and CNCM I-4702 deposited with the Collection Nationale de Cultures de Microorganismes [French National Collection of Microorganism Cultures] of the Institut Pasteur on Apr. 14, 2011 and Nov. 22, 2012, respectively.

4- The process according to claim 1, wherein the limitation of the growth rate of the microalga is obtained by reducing or exhausting trace elements in the fermentation medium or by limiting the O₂ transfer.

5- The process it according to claim 1, further comprising harvesting the biomass, optionally preparing a cell extract or lysate from this biomass, and then optionally extracting a DHA-rich crude oil.

6- The process according to claim 1, wherein the biomass obtained comprises at least 45% of DHA by weight of total fatty acids.

7- The process according to claim 1, wherein the biomass obtained comprises at least 40% of proteins by weight of biomass (g/g) expressed in N.6.25, including at least 10% of arginine and at least 25% of glutamic acid by weight relative to total amino acids.

8- A process for producing a biomass, comprising,
culturing microalgae of genus Thraustochytrium in a fermentation medium to enrich docosahexaenoic acid (DHA), arginine and glutamic acid amino acid content,
feeding a source of nitrogen to the medium,
assessing μ/μmax during said culturing, wherein μ is the actual growth rate of the microalga and μmax is the is optimal growth rate of the microalgae,
maintaining or adding said nitrogen source to the fermentation medium when μ/μmax of the microalgae becomes less than 0.2,
optionally, controlling the supply oxygen to the fermentation medium such that μ drops to μ/μmax <0.2 rapidly, and
recovering a biomass, including at least 10% of arginine and at least 25% of glutamic acid by weight relative to total amino acid content.

9- The process according to claim 8, wherein the microalgae is one of Schizochytrium sp. Strain CNCM I-4469, or Schizochytrium mangrovei strain CNCM I-4702.

10- The process according to claim 1, wherein the biomass includes at least 25% of DHA by weight of total fatty acids.

11- The process according to claim 9, wherein the biomass includes at least 45% of DHA by weight of total fatty acids.

12- The process according to claim 1, wherein the biomass includes at 15% of arginine and at least 40% of glutamic acid.

13- The process according to claim 9, wherein the biomass includes at 15% of arginine and at least 40% of glutamic acid.


SEQUENCE LISTING (2 sequences)

SEQ ID NO: 1 - DNA, 479 bp, Schizochytrium sp
gagggttttacattgctctcattccaatagcaagacgcgaagcgccccgcattgatatttctcgtcactacctcgtggagtccacattgggtaatttacgcgcctgctgccttccttggatgtggtagccgtctctcaggctccctctccggagtcgagccctaactccccgtcacccgttatagtcaccgtaggccaataccctaccgtcgacaactgatggggcagaaactcaaacgattcatcgctccgaaaagcgatctgctcaattatcatgactcaccaagagagttggcttagacctaataagtgcggccctccccgaaagtcgggcccgtacagcacgtattaattccagaattactgcaggtatccgtataaaggaactaccgaagggattataactgatataatgagccgttcgcagtttcacagtataattcgcttatacttacacatgcatggcttagtctttgaga

SEQ ID NO: 2 - DNA, 454 bp, Schizochytrium mangrovei
ggttttacattgctctcattccgatagcaaaacgcatacacgcttcgcatcgatatttctcgtcctacctcgtggagtccacagtgggtaatttacgcgcctgctgctatccttggatatggtagccgtctctcaggctccctctccggagtcgagccctaactctccgtcacccgttatagtcaccgtagtccaatacactaccgtcgacaactgatggggcagaaactcaaacgattcatcgaccaaaawagtcaatctgctcaattatcatgattcaccaataaaatcggcttcaatctaataagtgcagccccatacagggctcttacagcatgtattatttccagaattactgcaggtatccatataaaagaaactaccgaagaaattattactgatataatgagccgttcgcagtctcacagtacaatcgcttatacttacacagcag